(19) United States
(12) Patent Application Publication
(10) Pub. No.: US 2025/0375313 A1
(43) Pub. Date: Dec. 11, 2025

(54) HEAD THERMOREGULATORY APPARATUS

(71) Applicants: Graham Grove, Calistoga, CA (US); Thalia Alfaro, Napa, CA (US); Carla Alvarado Negrete, Calistoga, CA (US); Daisy Avina Hernandez, Calistoga, CA (US); Logan Brooks, Calistoga, CA (US); Troy Brooks, Calistoga, CA (US); Cesar Chino, Calistoga, CA (US); Zeneb Cortez-Arroyo, Santa Rosa, CA (US); Bernardo Cruz, Calistoga, CA (US); Karina Diaz, Calistoga, CA (US); Bianca Diaz De Niz, Calistoga, CA (US); Elias Escobar Argueta, Santa Rosa, CA (US); Violet Grove, Calistoga, CA (US); Raul Guerrero Parada, Calistoga, CA (US); Litzy Hernandez Avina, Calistoga, CA (US); Arlette Hernandez-Quevedo, Middletown, CA (US); Aaron Heth, Calistoga, CA (US); Landon Kohler, Calistoga, CA (US); Cindy Lopez, Calistoga, CA (US); Xitlali Lopez-Quirino, Calistoga, CA (US); Jose Montanez, Calistoga, CA (US); Kelly Montanez, Calistoga, CA (US); Alexa Navarrete-Avila, Calistoga, CA (US); Daisy Robles, Calistoga, CA (US); Ana Rosete, Calistoga, CA (US); Jimena Rubio Arias, Calistoga, CA (US); Guadalupe Rubio Pacheco, Calistoga, CA (US); Jennifer Servin-Flores, Calistoga, CA (US); Jayden Sibbu, Calistoga, CA (US); Beatrix Webb, Calistoga, CA (US)

(72) Inventors: Graham Grove, Calistoga, CA (US); Thalia Alfaro, Napa, CA (US); Carla Alvarado Negrete, Calistoga, CA (US); Daisy Avina Hernandez, Calistoga, CA (US); Logan Brooks, Calistoga, CA (US); Troy Brooks, Calistoga, CA (US); Cesar Chino, Calistoga, CA (US); Zeneb Cortez-Arroyo, Santa Rosa, CA (US); Bernardo Cruz, Calistoga, CA (US); Karina Diaz, Calistoga, CA (US); Bianca Diaz De Niz, Calistoga, CA (US); Elias Escobar Argueta, Santa Rosa, CA (US); Violet Grove, Calistoga, CA (US); Raul Guerrero Parada, Calistoga, CA (US); Litzy Hernandez Avina, Calistoga, CA (US); Arlette Hernandez-Quevedo, Middletown, CA (US); Aaron Heth, Calistoga, CA (US); Landon Kohler, Calistoga, CA (US); Cindy Lopez, Calistoga, CA (US); Xitlali Lopez-Quirino, Calistoga, CA (US); Jose Montanez, Calistoga, CA (US); Kelly Montanez, Calistoga, CA (US); Alexa Navarrete-Avila, Calistoga, CA (US); Daisy Robles, Calistoga, CA (US); Ana Rosete, Calistoga, CA (US); Jimena Rubio Arias, Calistoga, CA (US); Guadalupe Rubio Pacheco, Calistoga, CA (US); Jennifer Servin-Flores, Calistoga, CA (US); Jayden Sibbu, Calistoga, CA (US); Beatrix Webb, Calistoga, CA (US)

(21) Appl. No.: 18/737,161

(22) Filed: Jun. 7, 2024

(51) Int. Cl. A61F 7/00; A61B 5/01

(52) U.S. Cl. CPC A61F 7/00; A61B 5/01; A61F 2007/0007

(57) ABSTRACT

Head thermoregulatory apparatuses, related devices and methods, are disclosed herein. In some embodiments, a head thermoregulatory apparatus may include a core body temperature sensor; a head thermoregulatory device including a fluid pad, an input port, and an output port; a fluid device configured to heat or cool a fluid, the fluid device including the fluid, an input port, and an output port; fluid hoses between the head thermoregulatory device and the fluid device; a fluid valve coupled to the fluid hoses; and control circuitry coupled to the fluid valve and to the core body temperature sensor, wherein the control circuitry is configured to activate the fluid valve based on a determination that an absolute difference between a subsequent core body temperature and a baseline core body temperature exceeds a threshold. In some embodiments, the fluid device cools the fluid. In other embodiments, the fluid device heats the fluid.

### BACKGROUND

The present disclosure relates to personal thermoregulatory devices. More particularly, this disclosure relates to wearable personal cooling and/or heating apparatus for regulating a core body temperature of a user.

### BRIEF DESCRIPTION OF THE DRAWINGS

Embodiments will be readily understood by the following detailed description in conjunction with the accompanying drawings. To facilitate this description, like reference numerals designate like structural elements. Embodiments are illustrated by way of example, and not by way of limitation, in the figures of the accompanying drawings.

FIG. 1 is a schematic front view of an example head thermoregulatory device of a thermoregulatory apparatus according to some embodiments of the present disclosure.

FIG. 2 is a schematic side view of an example head thermoregulatory device of a thermoregulatory apparatus according to some embodiments of the present disclosure.

FIG. 3 is a schematic perspective view of an example head thermoregulatory device of a thermoregulatory apparatus according to some embodiments of the present disclosure.

FIG. 4 is a schematic side view of an example fluid thermoregulatory device of a thermoregulatory apparatus according to some embodiments of the present disclosure.

FIG. 5 is a schematic back view of an example fluid thermoregulatory device of a thermoregulatory apparatus according to some embodiments of the present disclosure.

FIG. 6 is a magnified schematic side view of an example fluid thermoregulatory device of a thermoregulatory apparatus according to some embodiments of the present disclosure.

FIG. 7 is a schematic front view of an example core body temperature sling according to some embodiments of the present disclosure.

FIG. 8 is a schematic diagram of a fluid system of an example thermoregulatory apparatus according to some embodiments of the present disclosure.

FIG. 9 is a schematic flow diagram listing example operations that may be associated with thermoregulation of a core body temperature according to some embodiments of the present disclosure.

### DETAILED DESCRIPTION

The regulation of core body temperature is crucial for maintaining physiological homeostasis and overall health. Core body temperature refers to the internal temperature of the body, which is typically maintained within a narrow range despite external environmental conditions. In hot environmental conditions, the human body can experience hyperthermia, where the core temperature rises to dangerously high levels, leading to heat stress, heat exhaustion, or heat stroke. Conversely, in cold environmental conditions, the body can suffer from hypothermia, where the core temperature drops below the normal range, resulting in shivering, reduced motor function, and potentially life-threatening conditions.

In many occupations, a person may be exposed to extreme environmental conditions including hot and/or cold temperatures. For example, wildland firefighters, who typically wear up to 40 pounds of personal protective equipment, may be exposed to significantly elevated temperatures while fighting a wildfire. In fact, numerous line-of-duty deaths of firefighters may be attributed to overexertion and heat stress. In another example, agricultural workers may be exposed to heat stress or cold stress when working in the fields. Agricultural field workers may experience significantly higher heat-related or cold-related mortality rate compared to workers from other industries.

Traditional methods of thermoregulation, such as wearing additional layers of clothing in cold environments or using fans and air conditioning in hot environments, have significant limitations. For instance, adding or removing clothing layers can be inconvenient and may not provide the rapid response needed to prevent thermal discomfort or medical emergencies. Similarly, stationary cooling and heating devices are not always accessible, practical, or effective in outdoor or mobile scenarios.

Existing wearable devices designed to assist with thermoregulation, such as cooling vests or heated garments, also present several challenges. These devices often rely on bulky and heavy materials, which can restrict movement and reduce comfort. Furthermore, the efficiency of these devices in effectively regulating core body temperature can be inconsistent, as they may not provide uniform heating or cooling across the body.

Another significant issue with current wearable thermoregulatory devices is their inability to adapt to rapidly changing environmental conditions. For instance, a user transitioning from an air-conditioned indoor environment to a hot outdoor setting may experience a sudden and uncomfortable temperature shift. Existing devices often lack the capability to dynamically adjust their thermal output in response to such changes, leading to periods of thermal discomfort.

Moreover, many wearable thermoregulatory solutions do not adequately address the need for user-specific customization. Individual variations in metabolism, activity level, and personal comfort preferences mean that a one-size-fits-all approach is often ineffective. The inability to tailor the thermal regulation to the specific needs of the user can result in suboptimal performance and user dissatisfaction.

In summary, there is a clear need for an improved wearable thermoregulatory device that can effectively and efficiently regulate core body temperature in hot and/or cold environmental conditions. Such a device should offer enhanced comfort, mobility, adaptability, and user-specific customization to address the limitations of current solutions.

The thermoregulatory cooling apparatus disclosed herein is a portable, lightweight, and affordable smart cooling device designed for firefighters battling wildland fires in remote areas. This device is programmed to help keep firefighters cool and prevent heat stress by monitoring both the firefighter's core body temperature and the external air temperature (e.g., environmental temperature). It features a small microcontroller and a power source, such as a battery, connected to temperature sensors that track these temperatures. When dangerous levels are detected, the microcontroller triggers alarms, such as colored lights within the firefighter's line of sight, to alert them.

The cooling mechanism of the apparatus is automated and works by pumping fluid through a cooling device and flowing the cold fluid to a head cooling device, for a specific interval, based on a change in the firefighter's core body temperature. In particular, the cold fluid circulates through a membrane located on the forehead, which is worn inside head gear, such as a helmet, and may or may not be directly attached to it. For hygiene and comfort, the membrane may be surrounded by a removable, washable thin cotton layer. The cooling apparatus may further include emergency safety release clasps on the carrying bag for quick removal, if necessary. To avoid interference with other equipment, all cooling system tubing may run down the right side of the neck and shoulder and features easy disconnect systems for convenience.

The thermoregulatory cooling and heating apparatus disclosed herein is a portable and affordable SMART cooling and heating device designed for workers, such as agricultural or construction workers, to wear while working outdoors. It functions to keep a worker cool in hot weather and warm in cold weather, helping to prevent heat stress or hypothermia and associated discomfort. The device includes a small microcontroller and a battery connected to a temperature sensor that monitors the worker's core body temperature. When the core body temperature approaches a critical high or low level the microcontroller activates colored light alarms within the worker's line of sight to alert them.

The cooling and heating functions of the apparatus are automated and work by pumping fluid through a cooling or heating device and flowing the cold or warm fluid to a head cooling device, for a specific interval, based on a change in the worker's core body temperature. In particular, the fluid circulates through a membrane located on the forehead, which may be worn inside a hat and may or may not be directly attached to it. For hygiene and comfort, the membrane may be surrounded by a removable, washable thin cotton layer. This ensures that outdoor workers can maintain a safe and comfortable body temperature while working in varying weather conditions.

Each of the apparatuses, devices, and methods of the present disclosure may have several innovative aspects, no single one of which is solely responsible for all the desirable attributes disclosed herein. Details of one or more implementations of the subject matter described in this specification are set forth in the description below and the accompanying drawings.

In the following detailed description, various aspects of the illustrative implementations may be described using terms commonly employed by those skilled in the art to convey the substance of their work to others skilled in the art.

The terms “circuit” and “circuitry” mean one or more passive and/or active electrical and/or electronic components that are arranged to cooperate with one another to provide a desired function. The terms also refer to analog circuitry, digital circuitry, hard wired circuitry, programmable circuitry, microcontroller circuitry and/or any other type of physical hardware electrical and/or electronic component.

The terms “substantially,” “close,” “approximately,” “near,” and “about,” generally refer to being within +/−20% of a target value (e.g., within +/−5% or 10% of a target value) based on the context of a particular value as described herein or as known in the art.

The description uses the phrases “in an embodiment” or “in some embodiments,” which may each refer to one or more of the same or different embodiments.

Furthermore, the terms “comprising,” “including,” “having,” and the like, as used with respect to embodiments of the present disclosure, are synonymous.

The disclosure may use perspective-based descriptions such as “above,” “below,” “top,” “bottom,” and “side”; such descriptions are used to facilitate the discussion and are not intended to restrict the application of disclosed embodiments.

The term “between,” when used with reference to measurement ranges, is inclusive of the ends of the measurement ranges.

For the purposes of the present disclosure, the phrase “A and/or B” means (A), (B), or (A and B). For the purposes of the present disclosure, the phrase “A, B, and/or C” means (A), (B), (C), (A and B), (A and C), (B and C), or (A, B, and C).

Although certain elements may be referred to herein in the singular forms “a,” “an,” and “the” include plural forms.

Unless otherwise specified, the use of the ordinal adjectives “first,” “second,” and “third,” etc., to describe a common object, merely indicate that different instances of like objects are being referred to, and are not intended to imply that the objects so described must be in a given sequence, either temporally, spatially, in ranking or in any other manner.

In the following detailed description, reference is made to the accompanying drawings that form a part hereof, and in which is shown, by way of illustration, embodiments that may be practiced. It is to be understood that other embodiments may be utilized, and structural or logical changes may be made without departing from the scope of the present disclosure. Therefore, the following detailed description is not to be taken in a limiting sense.

The accompanying drawings are not necessarily drawn to scale.

In the drawings, same reference numerals refer to the same or analogous elements/materials shown so that, unless stated otherwise, explanations of an element/material with a given reference numeral provided in context of one of the drawings are applicable to other drawings where element/materials with the same reference numerals may be illustrated. Further, the singular and plural forms of the labels may be used with reference numerals to denote a single one and multiple ones respectively of the same or analogous type, species, or class of element.

In the drawings, a particular number and arrangement of structures and components are presented for illustrative purposes and any desired number or arrangement of such structures and components may be present in various embodiments.

Various operations may be described as multiple discrete actions or operations in turn in a manner that is most helpful in understanding the claimed subject matter. However, the order of description should not be construed as to imply that these operations are necessarily order dependent. In particular, these operations may not be performed in the order of presentation. Operations described may be performed in a different order from the described embodiment or may be performed more than once. Various additional operations may be performed, and/or described operations may be omitted in additional embodiments.

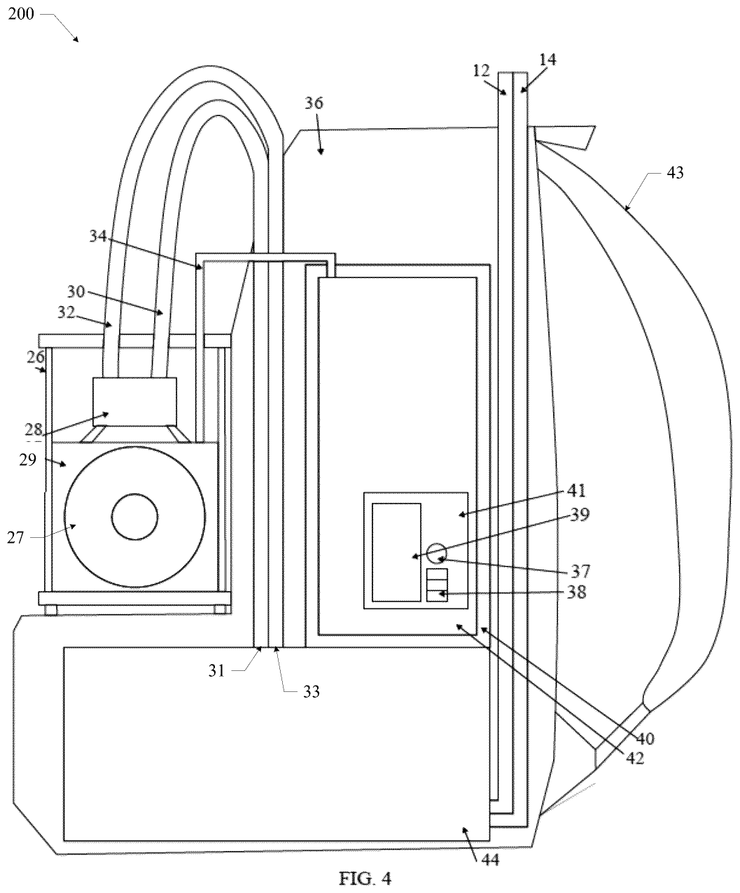

FIG. 1 is a schematic front view of an example head thermoregulatory device of a thermoregulatory apparatus according to some embodiments of the present disclosure. As shown in FIG. 1, a head thermoregulatory device 100 may include a fluid pad 20, a fluid hose 14 coupled to an input port 13, and a fluid hose 12 coupled to an output port 11. The fluid pad 20 may be positioned in a head mount 10 to contact a forehead of a user 22. A head mount 10 may include any suitable means for attaching the fluid pad 20 to the user's forehead, including, for example, a hat, a helmet, a skullcap, or a cranial strap. In some embodiments, as shown in FIG. 3, a head mount 10 may include a headband 24. A fluid may be maintained at a therapeutic temperature (e.g., by a fluid device 200, as shown in FIG. 4) to thermoregulate a core body temperature of a user 22 through the fluid pad 20. In some embodiments, for example, when a user 22 is experiencing hot environmental conditions, the fluid may be cooled and targeted to be maintained at a temperature between 58 degrees Fahrenheit and 70 degrees Fahrenheit. In some embodiments, for example, when a user 22 is experiencing cold environmental conditions, the fluid may be cooled and targeted to be maintained at a temperature between 98.6 degrees Fahrenheit and 109 degrees Fahrenheit. Fluid hoses 12, 14 may be coupled to the fluid pad 20 and to the fluid device 200 forming a fluid circulation loop, such that the fluid may be circulated between the fluid device 200 and the fluid pad 20. In some embodiments, the fluid pad 20 may include a flexible pouch for containing the fluid, such as intravenous (IV) bag, and made be made of any suitable material, such as a polyvinyl chloride (PVC), ethylene vinyl acetate (EVAM), polypropylene, copolyester ether, or another waterproof thermally conductive material. A thermally conductive flexible material is preferred to provide maximum surface contact for thermoregulation. A fluid may include a liquid or a gas, and may include any suitable fluid, such as water. The fluid hose 12, 14 may include any suitable conduit for flowing the liquid, such as polyurethane tubing. In some embodiments, the fluid pad 20 may be encased in a lightweight washable fabric, such as cotton.

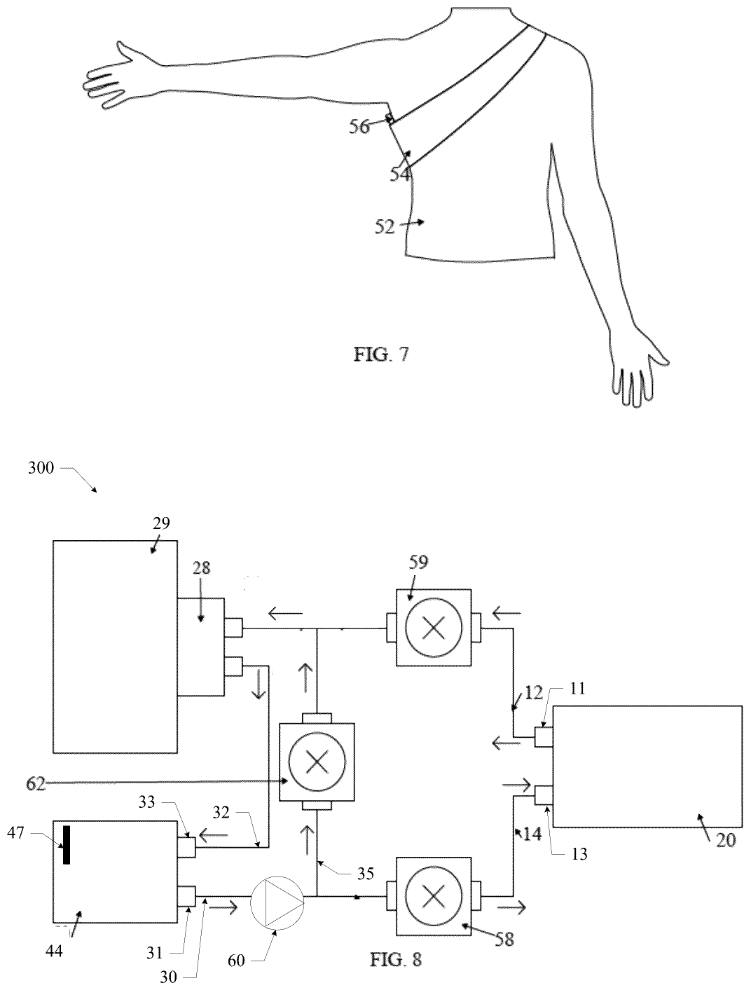

A head thermoregulatory device 100 may further include one or more warning elements 16, 17. Warning elements 16, 17 may be attached to the head mount 10 by connector 18. Warning elements 16, 17 may include any suitable component for alerting a user 22 to unsafe conditions, including, for example, a visual indicator, such as a light, an auditory indicator, such as a siren or alarm, or haptic feedback. For example, when a user 22 is experiencing hot environmental conditions, control circuitry may be coupled to a core body temperature sensor (e.g., a core body temperature sensor 56, as shown in FIG. 7) and to warning element 16, where the control circuitry is configured to activate the warning element 16 based on a determination that the core body temperature sensor 56 is detecting a core body temperature greater than or equal to 102 degrees Fahrenheit. In another example, when a user 22 is experiencing hot environmental conditions, head thermoregulatory device 100 may further include an environmental temperature sensor 19, and control circuitry may be coupled to the environmental temperature sensor and to warning element 17, where the control circuitry is configured to activate the warning element 17 based on a determination that the environmental temperature sensor 19 is detecting an environmental temperature equal to or greater than 150 degrees Fahrenheit. In yet another example, when a user 22 is experiencing cold environmental conditions, control circuitry coupled to warning element 16 may be configured to activate the warning element 16 based on a determination that the core body temperature sensor 56 is detecting a core body temperature greater than or equal to 102 degrees Fahrenheit, and control circuitry coupled to warning element 17 may be configured to activate the warning element 17 based on a determination that the core body temperature sensor 56 is detecting a core body temperature less than or equal to 95 degrees Fahrenheit. Although FIG. 1 shows a particular placement of warning elements 16, 17, the placement of a warning element 16, 17 may depend on the type of indicator. Although FIG. 1 shows two warning elements 16, 17, a head thermoregulatory device 100 may include any suitable number of warning elements, including none, one, or more than two. Although FIG. 1 shows a particular placement of the environmental temperature sensor 19, the environmental temperature sensor 19 may be placed in any suitable location.

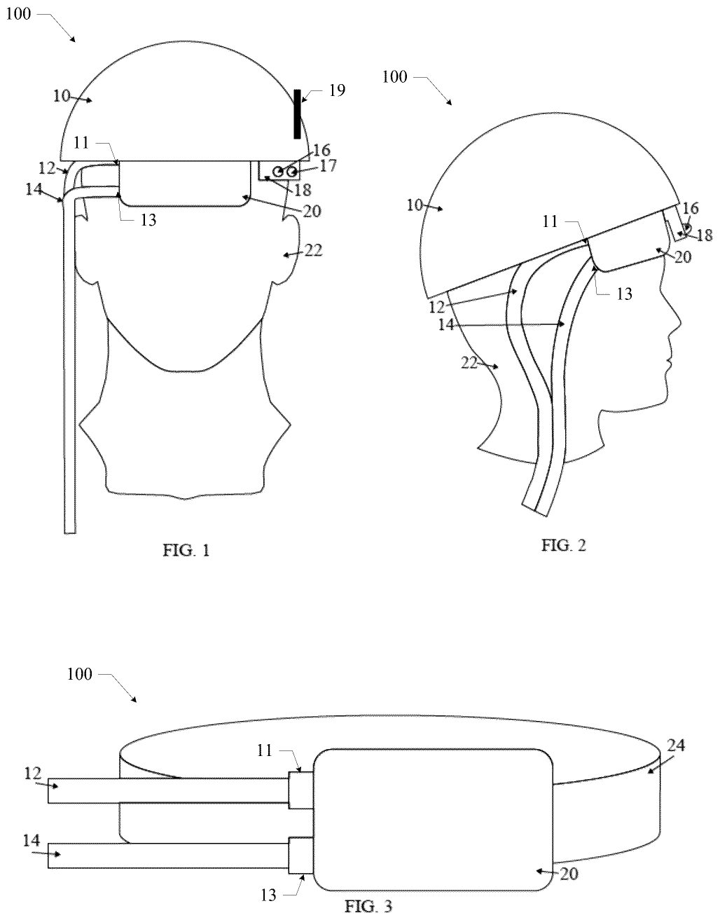

FIG. 2 is a schematic side view of an example head thermoregulatory device of a thermoregulatory apparatus according to some embodiments of the present disclosure. The configuration of the embodiment shown in the figure is like that of FIG. 1, except for differences as described further. The configuration of FIG. 2 further illustrates fluid hoses 12, 14 collected along a right side of a neck and shoulder of a user 22 to minimize interference with other user equipment. In other embodiments, fluid hoses 12, 14 may be positioned in another way. In some embodiments, fluid hoses 12, 14 may further include easy disconnect systems for safety and convenience.

FIG. 3 is a schematic perspective view of an example head thermoregulatory device of a thermoregulatory apparatus according to some embodiments of the present disclosure. The configuration of the embodiment shown in the figure is like that of FIG. 1, except for differences as described further. The configuration of FIG. 3 further illustrates a headband 24 as a head mount (e.g., the head mount 10, as shown in FIG. 1) for the fluid pad 20, fluid hose 14 coupled to an input port 13, and fluid hose 12 coupled to an output port 11.

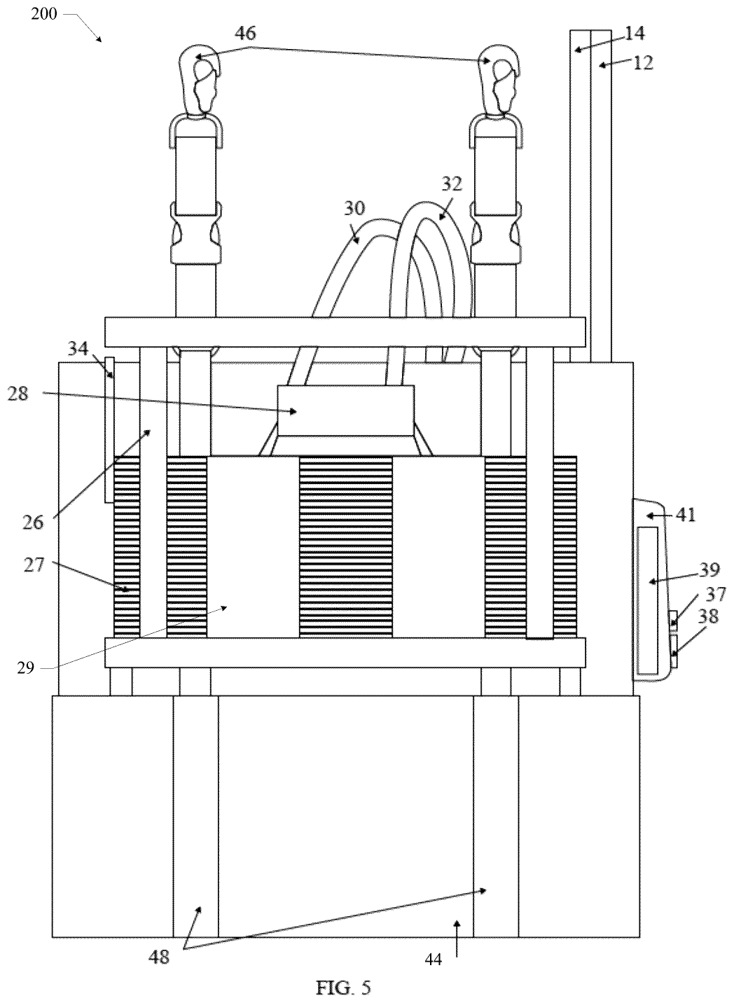

FIG. 4 is a schematic side view of an example fluid thermoregulatory device of a thermoregulatory apparatus according to some embodiments of the present disclosure. As shown in FIG. 4, a fluid thermoregulatory device 200 may include a fluid reservoir 44, a thermoelectric cooler (TEC) 28, a heat sink 29, a fan 27, fluid hose 30 coupled to an output port 31, and fluid hose 32 coupled to an input port 33 for maintaining the fluid in the fluid reservoir 44 at a therapeutic temperature. In some embodiments, the TEC 28, the heat sink 29, and the fan 27 may be positioned within a frame 26 or cage on the outside of the carrying apparatus 36 to allow the fan 27 to efficiently flow hot or cold air away from the heat sink 29. Fluid hoses 30, 32 may be coupled to the fluid reservoir 44 and to the TEC 28 forming a fluid circulation loop, such that the fluid may be circulated between the fluid reservoir 44 and the TEC 28. Fluid hose 30 may be in thermal contact with the TEC 28, such that the fluid may be cooled or heated to a therapeutic temperature. The fluid thermoregulatory device 200 may further include control circuitry connected to a switch 38 configured to change a polarity of the TEC 28 based on whether the fluid is cooled to a therapeutic temperature or heated to a therapeutic temperature. The fluid may be circulated between the reservoir 44 and the fluid pad 20 (e.g., as shown in FIG. 1) via fluid hoses 12, 14. The fluid thermoregulatory device 200 may include a bundle of electrical wires 34 electrically coupling the TEC 28 and the fan 27 to an electrical box 42. In some embodiments, the electrical box 42 may be protected by waterproofing 40. The electrical box 42 may further include a power button 37 for turning the fluid thermoregulatory device 200 on and off, and a power source 39, such as a battery pack. The fluid thermoregulatory device 200 may be contained in a carrying apparatus 36. The carrying apparatus 36 may include any suitable means for wearing the carrying apparatus 36, including a strap 43, as shown, a buckle, a belt, or a fastener (e.g., a fastener 46, as shown in FIG. 5). The power button 37, the switch 38, and the power source 39 may be positioned to be easily accessible, such as in an external pocket 41 of the carrying apparatus 36.

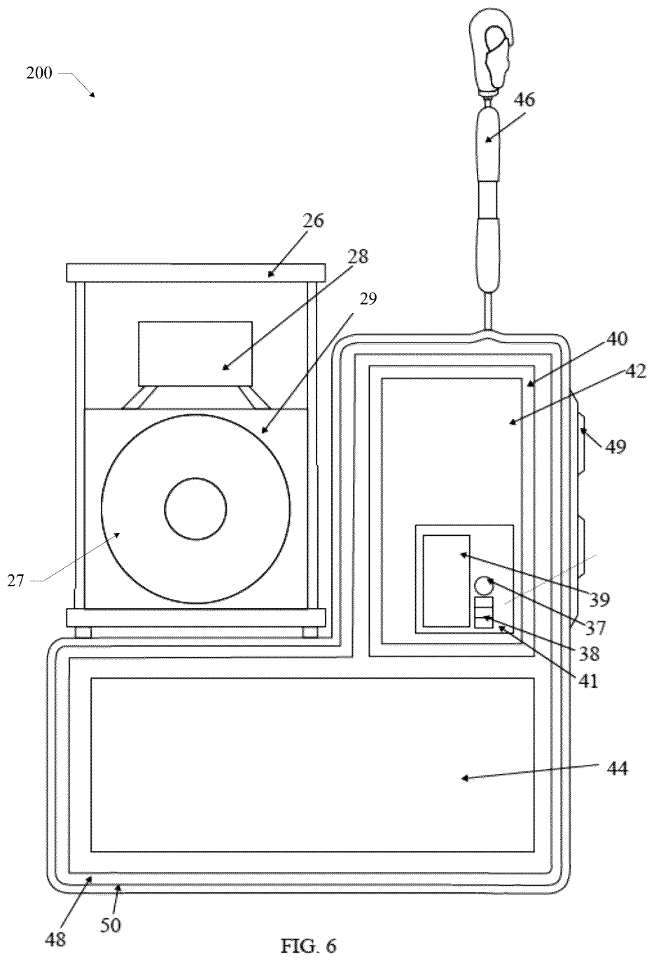

FIG. 5 is a schematic back view of an example fluid thermoregulatory device of a thermoregulatory apparatus according to some embodiments of the present disclosure. As shown in FIG. 5, a fluid thermoregulatory device 200 may include a heat sink 29 thermally coupled to the TEC 28 for maintaining the fluid in the fluid reservoir 44 at a therapeutic temperature. The heat sink 29 may include one or more fins and one or more fans 27 adjacent to or in between the one or more fins. The fluid thermoregulatory device 200 may further include fasteners 46 and straps 48 coupled to the fasteners 46 for securing the fluid thermoregulatory device 200 to other packs or equipment worn by a user. In some embodiments, the fasteners 46 may include a snap hook or a carabiner. In some embodiments, the fluid thermoregulatory device 200 or a portion of the fluid thermoregulatory device 200 (e.g., the reservoir 44) may be contained in a fabric pouch (e.g., the fabric pouch 50, as shown in FIG. 6) and the straps 48 may wrap around the fabric pouch. In some embodiments, the fabric pouch may include a fire resistant material.

FIG. 6 is a magnified schematic side view of an example fluid thermoregulatory device of a thermoregulatory apparatus according to some embodiments of the present disclosure. The configuration of the embodiment shown in the figure is like that of FIG. 5, except for differences as described further. The configuration of FIG. 6 further illustrates a webbing material 49 attached to the fabric pouch 50 holding the reservoir 44 and the electrical box 42. The webbing material 49 may be used to attach the fluid thermoregulatory device 200 to other equipment worn by a user.

FIG. 7 is a schematic front view of an example core body temperature sling according to some embodiments of the present disclosure. As shown in FIG. 7, a thermoregulatory apparatus may further include a sling 54 with a core body temperature sensor 56 that wraps around a user's torso 52. The sling 54 may include a means for positioning and maintaining the core body temperature sensor 56 in an armpit of the user to provide accurate core body temperature readings. The core body temperature sensor 56 may be coupled to control circuitry and may send a signal indicating a core body temperature. A baseline core body temperature may be established once a core body temperature reaches an equilibrium, which in some embodiments, may be between four (4) minutes and eight (8) minutes after powering on a thermoregulatory apparatus. A signal indicating a subsequent core body temperature may be sent based on a predetermined time duration, for example, every two (2) seconds, every five (5) seconds, or every ten (10) seconds. The signal indicating a subsequent core body temperature may be used to determine whether to activate different elements of the thermoregulatory apparatus, for example, warning elements (e.g., warning 16, 17 of FIG. 1), and elements of the fluid system, as described below with reference to FIG. 8.

FIG. 8 is a schematic diagram of a fluid system of an example thermoregulatory apparatus according to some embodiments of the present disclosure. As shown in FIG. 8, a fluid system 300 may include a first fluid circulation loop between a fluid thermoregulatory device 200 (e.g., as shown in FIG. 4) and a head thermoregulatory device 100 (e.g., as shown in FIG. 1) for the thermoregulation of a core body temperature of a user, and a second fluid circulation loop within the fluid thermoregulatory device 200 for maintaining the fluid at a therapeutic temperature. A fluid system 300 may include a fluid pad 20 with an input port 13 and an output port 11, a fluid reservoir 44 with an input port 33 and an output port 31, a TEC 28 thermally coupled to a heat sink 29, a pump 60, fluid valves 58, 59, 62, and fluid hoses 12, 14, 30, 32, 35.

Fluid hoses 12, 14, 30, 32 connecting the reservoir 44, the pump 60, fluid valve 58, fluid pad 20, fluid valve 59, and TEC 28 may form the first circulation loop. Control circuitry may be coupled to fluid valves 58, 59 and may be configured to activate (e.g., open) fluid valves 58, 59 based on a determination that an absolute difference between a subsequent core body temperature and a baseline core body temperature exceeds a threshold, as described above with reference to FIG. 7. The control circuitry may be configured to deactivate (e.g., close) fluid valves 58, 59 after a pre-determined period of time, for example, fluid valves 58, 59 may be deactivated three (3) minutes after activation.

Fluid hoses 30, 32, 35 connecting the reservoir 44, the pump 60, fluid valve 62, and TEC 28 may form the second circulation loop. Control circuitry may be coupled to fluid valve 62 and may be configured to activate fluid valve 62 when the thermoregulatory apparatus is turned on and when the fluid valves 58, 59 are deactivated, and may be configured to deactivate fluid valve 62 when the fluid valves 58, 59 are activated. The fluid may be circulated through the first fluid circulation loop and the second fluid circulation loop by the pump 60. Control circuitry also may be coupled to the pump 60 and a fluid temperature sensor 47 that measures a temperature of the fluid in the reservoir 44 to turn the pump 60 on to heat or cool the fluid to a therapeutic target temperature (e.g., as described above with reference to FIG. 1), and turn the pump 60 off when the therapeutic target temperature is met. Although FIG. 8 shows a particular number and arrangement of components, a fluid system 300 may include any suitable number and arrangement of components.

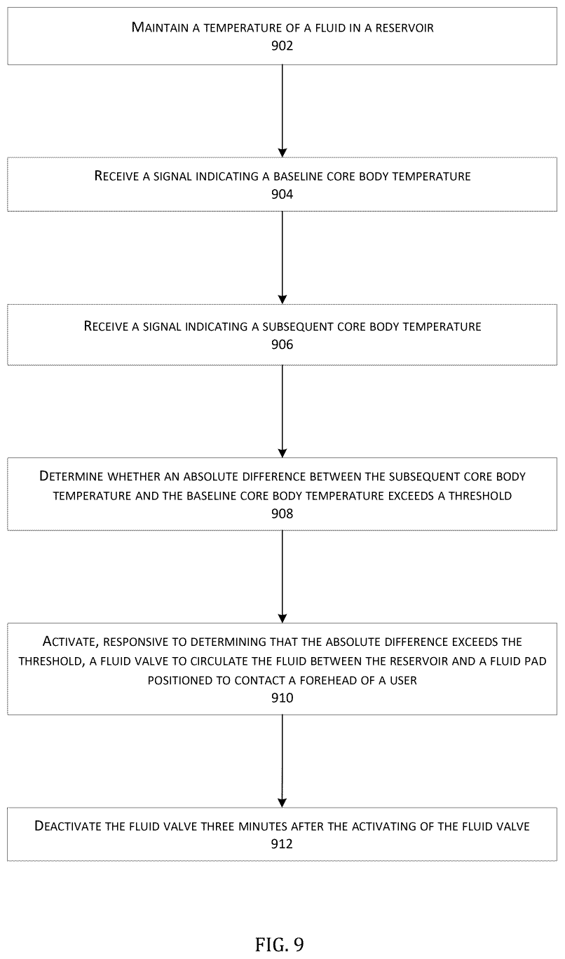

FIG. 9 is a flow diagram of an example method for thermoregulation of a core body temperature according to some embodiments of the present disclosure. At 902, maintain a temperature of a fluid in a reservoir. A fluid may be maintained at a therapeutic temperature by a fluid device 200, as described above with reference to FIG. 8. At 904, receive a signal indicating a baseline core body temperature. At 906, receive a signal indicating a subsequent core body temperature. At 908, determine whether an absolute difference between the subsequent core body temperature and the baseline core body temperature exceeds a threshold. A threshold may be exceeded if the absolute difference is greater than or equal to 1 degree Fahrenheit. At 910, activate, responsive to determining that the absolute difference exceeds the threshold, a fluid valve to circulate the fluid between the reservoir and a fluid pad positioned to contact a forehead of a user. At 912, deactivate the fluid valve to stop circulating the fluid between the reservoir and a fluid pad three (3) minutes after the activating of the fluid valve. The operations described at 902 through 912 may be repeated for as long as the thermoregulatory apparatus is in use to thermoregulate a core body temperature of a user. In embodiments, where the fluid in the reservoir is being cooled, repeating the operations described at 910 through 912 may be delayed for a period of approximately five (5) minutes to reduce the risk of the user experiencing vasoconstriction. The operations described at 902 through 908 may be repeated to confirm that thermoregulation of a user's core body temperature is not required.

The above description of illustrated implementations of the disclosure, including what is described in the abstract, is not intended to be exhaustive or to limit the disclosure to the precise forms disclosed. While specific implementations of, and examples for, the disclosure are described herein for illustrative purposes, various equivalent modifications are possible within the scope of the disclosure, as those skilled in the relevant art will recognize.

The following paragraphs provide various examples of the embodiments disclosed herein.

Example 1 is a cooling apparatus, including a core body temperature sensor; a head cooling device including a fluid pad, an input port, and an output port; a fluid cooling device including a fluid, an input port, and an output port; fluid hoses between the head cooling device and the fluid cooling device; a fluid valve coupled to the fluid hoses; and control circuitry coupled to the fluid valve and to the core body temperature sensor, wherein the control circuitry is configured to activate the fluid valve based on a determination that a difference between a subsequent core body temperature and a baseline core body temperature exceeds a threshold.

Example 2 may include the subject matter of Example 1, and may further specify that the threshold is exceeded if the difference is greater than or equal to 1 degree Fahrenheit.

Example 3 may include the subject matter of Example 1, and may further include a carrying apparatus containing the fluid cooling device and a means for wearing the carrying apparatus.

Example 4 may include the subject matter of Example 3, and may further specify that the means for wearing the carrying apparatus includes a strap, a belt, a fastener, or a buckle.

Example 5 may include the subject matter of Example 4, and may further specify that the fastener includes a snap hook or a carabiner.

Example 6 may include the subject matter of Example 1, and may further specify that the fluid pad is positioned in a head mount to contact a forehead of a user.

Example 7 may include the subject matter of Example 6, and may further specify that the head mount includes a headband, a hat, a skullcap, or a cranial strap.

Example 8 may include the subject matter of Example 1, and may further specify that the fluid hoses are first fluid hoses forming a first fluid circulation loop, and the fluid cooling device further includes a fluid reservoir; a thermoelectric cooler (TEC); a heatsink thermally coupled to the TEC; second fluid hoses between the fluid reservoir and the TEC forming a second fluid circulation loop; and a pump for circulating the fluid through the first fluid circulation loop and the second fluid circulation loop.

Example 9 may include the subject matter of Example 8, and may further specify that the heatsink includes one or more fins; and a fan adjacent to or in between the one or more fins.

Example 10 may include the subject matter of Example 1, and may further include a sling including a means for positioning and maintaining the core body temperature sensor in an armpit of a user.

Example 11 may include the subject matter of Example 1, and may further include a warning element; and control circuitry coupled to the warning element and to the core body temperature sensor, wherein the control circuitry is configured to activate the warning element based on a determination that the core body temperature sensor is detecting a core body temperature greater than or equal to 102 degrees Fahrenheit.

Example 12 may include the subject matter of Example 11, and may further specify that the warning element includes a visual indicator, an auditory indicator, or haptic feedback.

Example 13 may include the subject matter of Example 1, and may further include an environmental temperature sensor; a warning element; and control circuitry coupled to the warning element and to the environmental temperature sensor, wherein the control circuitry is configured to activate the warning element based on a determination that the environmental temperature sensor is detecting an environmental temperature equal to or greater than 150 degrees Fahrenheit.

Example 14 may include the subject matter of Example 13, and may further specify that the warning element includes a visual indicator, an auditory indicator, or haptic feedback.

Example 15 is a thermoregulatory apparatus, including a core body temperature sensor; a head thermoregulatory device including a fluid pad, an input port, and an output port; a fluid device configured to heat or cool a fluid, the fluid device including the fluid, an input port, and an output port; fluid hoses between the head thermoregulatory device and the fluid device; a fluid valve coupled to the fluid hoses; and control circuitry coupled to the fluid valve and to the core body temperature sensor, wherein the control circuitry is configured to activate the fluid valve based on a determination that an absolute difference between a subsequent core body temperature and a baseline core body temperature exceeds a threshold.

Example 16 may include the subject matter of Example 15, and may further specify that the threshold is exceeded if the absolute difference is greater than or equal to 1 degree Fahrenheit.

Example 17 may include the subject matter of Example 15, and may further specify that the fluid device is configured to cool the fluid.

Example 18 may include the subject matter of Example 15, and may further specify that the fluid device is configured to heat the fluid.

Example 19 may include the subject matter of Example 15, and may further include a carrying apparatus containing the fluid device and a means for wearing the carrying apparatus.

Example 20 may include the subject matter of Example 19, and may further specify that the means for wearing the carrying apparatus includes a strap, a belt, a fastener, or a buckle.

Example 21 may include the subject matter of Example 15, and may further specify that the fluid pad is positioned in a head mount to contact a forehead of a user.

Example 22 may include the subject matter of Example 21, and may further specify that the head mount includes a headband, a hat, a skullcap, or a cranial strap.

Example 23 may include the subject matter of Example 15, and may further specify that the fluid hoses are first fluid hoses forming a first fluid circulation loop, and the fluid device further includes a fluid reservoir; a thermoelectric cooler (TEC); a heatsink thermally coupled to the TEC; second fluid hoses between the fluid reservoir and the TEC forming a second fluid circulation loop; and a pump for circulating the fluid through the first fluid circulation loop and the second fluid circulation loop.

Example 24 may include the subject matter of Example 23, and may further specify that the heatsink includes one or more fins; and a fan adjacent to or in between the one or more fins.

Example 25 may include the subject matter of Example 15, and may further include a sling including a means for positioning and maintaining the core body temperature sensor in an armpit of a user.

Example 26 may include the subject matter of Example 15, and may further include a warning element; and control circuitry coupled to the warning element and to the core body temperature sensor, wherein the control circuitry is configured to activate the warning element based on a determination that the core body temperature sensor is detecting a core body temperature greater than or equal to 102 degrees Fahrenheit.

Example 27 may include the subject matter of Example 26, and may further specify that the warning element includes a visual indicator, an auditory indicator, or haptic feedback.

Example 28 may include the subject matter of Example 15, and may further include a warning element; and control circuitry coupled to the warning element and to the core body temperature sensor, wherein the control circuitry is configured to activate the warning element based on a determination that the core body temperature sensor is detecting a core body temperature less than or equal to 95 degrees Fahrenheit.

Example 29 may include the subject matter of Example 28, and may further specify that the warning element includes a visual indicator, an auditory indicator, or haptic feedback.

Example 30 may include the subject matter of Example 15, and may further include an environmental temperature sensor; a warning element; and control circuitry coupled to the warning element and to the environmental temperature sensor, wherein the control circuitry is configured to activate the warning element based on a determination that the environmental temperature sensor is detecting an environmental temperature equal to or greater than 150 degrees Fahrenheit.

Example 31 may include the subject matter of Example 30, and may further specify that the warning element includes a visual indicator, an auditory indicator, or haptic feedback.

Example 32 is a method for thermoregulation of a core body temperature, including maintaining a temperature of a fluid in a reservoir; receiving a signal indicating a baseline core body temperature; receiving a signal indicating a subsequent core body temperature; determining whether an absolute difference between the subsequent core body temperature and the baseline core body temperature exceeds a threshold; activating, responsive to determining that the absolute difference exceeds the threshold, a fluid valve to circulate the fluid between the reservoir and a fluid pad positioned to contact a forehead of a user; and deactivating the fluid value three (3) minutes after the activating of the fluid valve.

Example 33 may include the subject matter of Example 32, and may further specify that the threshold is exceeded if the absolute difference is greater than or equal to 1 degree Fahrenheit

Example 34 may include the subject matter of Example 32, and may further specify that the temperature of the fluid in the reservoir is targeted to be maintained between 58 degrees Fahrenheit and 70 degrees Fahrenheit.

Example 35 may include the subject matter of Example 32, and may further specify that the temperature of the fluid in the reservoir is targeted to be maintained between Example 98.6 degrees Fahrenheit and 109 degrees Fahrenheit.

Example 36 may include the subject matter of Example 32, and may further specify that the temperature of the fluid is maintained by circulating the fluid, using a pump, through fluid hoses between the reservoir and a thermoelectric cooler (TEC).

Example 37 may include the subject matter of Example 32, and may further include determining whether the subsequent core body temperature is greater than or equal to 102 degrees Fahrenheit; and activating, responsive to determining the subsequent core body temperature is greater than or equal to 102 degrees Fahrenheit, a warning element.

Example 38 may include the subject matter of Example 32, and may further include determining whether the subsequent core body temperature is less than or equal to 95 degrees Fahrenheit; and activating, responsive to determining the subsequent core body temperature is less than or equal to 95 degrees Fahrenheit, a warning element.

Example 39 may include the subject matter of Example 32, and may further include receiving a signal indicating an environmental temperature; determining whether the environmental temperature is equal to or greater than 150 degrees Fahrenheit; and activating, responsive to determining the environmental temperature is equal to or greater than 150 degrees Fahrenheit, a warning element.

1. A cooling apparatus, comprising:
a core body temperature sensor;
a head cooling device including a fluid pad, an input port, and an output port;
a fluid cooling device including a fluid, an input port, and an output port;
fluid hoses between the head cooling device and the fluid cooling device;
a fluid valve coupled to the fluid hoses; and
control circuitry coupled to the fluid valve and to the core body temperature sensor, wherein the control circuitry is configured to activate the fluid valve based on a determination that a difference between a subsequent core body temperature and a baseline core body temperature exceeds a threshold.

2. The cooling apparatus of claim 1, wherein the threshold is exceeded if the difference is greater than or equal to 1 degree Fahrenheit.

3. The cooling apparatus of claim 1, further comprising:
a carrying apparatus containing the fluid cooling device and a means for wearing the carrying apparatus.

4. The cooling apparatus of claim 1, wherein the fluid pad is positioned in a head mount to contact a forehead of a user.

5. The cooling apparatus of claim 1, wherein the fluid hoses are first fluid hoses forming a first fluid circulation loop, and wherein the fluid cooling device comprises:
a fluid reservoir;
a thermoelectric cooler (TEC);
a heatsink thermally coupled to the TEC;
second fluid hoses between the fluid reservoir and the TEC forming a second fluid circulation loop; and
a pump for circulating the fluid through the first fluid circulation loop and the second fluid circulation loop.

6. The cooling apparatus of claim 1, further comprising:
a sling including a means for positioning and maintaining the core body temperature sensor in an armpit of a user.

7. The cooling apparatus of claim 1, further comprising:
an environmental temperature sensor;
a warning element; and
control circuitry coupled to the warning element and to the environmental temperature sensor, wherein the control circuitry is configured to activate the warning element based on a determination that the environmental temperature sensor is detecting an environmental temperature equal to or greater than 150 degrees Fahrenheit.

8. A thermoregulatory apparatus, comprising:
a core body temperature sensor;
a head thermoregulatory device including a fluid pad, an input port, and an output port, wherein the fluid pad is positioned to contact a forehead of a user;
a fluid device configured to heat or cool a fluid, the fluid device including the fluid, an input port, and an output port;
fluid hoses between the head thermoregulatory device and the fluid device;
a fluid valve coupled to the fluid hoses; and
control circuitry coupled to the fluid valve and to the core body temperature sensor, wherein the control circuitry is configured to activate the fluid valve based on a determination that an absolute difference between a subsequent core body temperature and a baseline core body temperature exceeds a threshold.

9. The thermoregulatory apparatus of claim 8, wherein the threshold is exceeded if the absolute difference is greater than or equal to 1 degree Fahrenheit.

10. The thermoregulatory apparatus of claim 8, wherein the fluid hoses are first fluid hoses forming a first fluid circulation loop, and wherein the fluid device comprises:
a fluid reservoir;
a thermoelectric cooler (TEC);
a heatsink thermally coupled to the TEC;
second fluid hoses between the fluid reservoir and the TEC forming a second fluid circulation loop; and
a pump for circulating the fluid through the first fluid circulation loop and the second fluid circulation loop.

11. The thermoregulatory apparatus of claim 8, further comprising:
a warning element; and
control circuitry coupled to the warning element and to the core body temperature sensor, wherein the control circuitry is configured to activate the warning element based on a determination that the core body temperature sensor is detecting a core body temperature greater than or equal to 102 degrees Fahrenheit.

12. The thermoregulatory apparatus of claim 8, further comprising:
a warning element; and
control circuitry coupled to the warning element and to the core body temperature sensor, wherein the control circuitry is configured to activate the warning element based on a determination that the core body temperature sensor is detecting a core body temperature less than or equal to 95 degrees Fahrenheit.

13. A method for thermoregulation of a core body temperature, comprising:
maintaining a temperature of a fluid in a reservoir;
receiving a signal indicating a baseline core body temperature;
receiving a signal indicating a subsequent core body temperature;
determining whether an absolute difference between the subsequent core body temperature and the baseline core body temperature exceeds a threshold;
activating, responsive to determining that the absolute difference exceeds the threshold, a fluid valve to circulate the fluid between the reservoir and a fluid pad positioned to contact a forehead of a user; and
deactivating the fluid value three (3) minutes after the activating of the fluid valve.

14. The method of claim 13, wherein the threshold is exceeded if the absolute difference is greater than or equal to 1 degree Fahrenheit.

15. The method of claim 13, wherein the temperature of the fluid in the reservoir is targeted to be maintained between 58 degrees Fahrenheit and 70 degrees Fahrenheit.

16. The method of claim 13, wherein the temperature of the fluid in the reservoir is targeted to be maintained between 98.6 degrees Fahrenheit and 109 degrees Fahrenheit.

17. The method of claim 13, wherein the temperature of the fluid is maintained by circulating the fluid, using a pump, through fluid hoses between the reservoir and a thermoelectric cooler (TEC).

18. The method of claim 13, further comprising:
determining whether the subsequent core body temperature is greater than or equal to 102 degrees Fahrenheit; and
activating, responsive to determining the subsequent core body temperature is greater than or equal to 102 degrees Fahrenheit, a warning element.

19. The method of claim 13, further comprising:
determining whether the subsequent core body temperature is less than or equal to 95 degrees Fahrenheit; and
activating, responsive to determining the subsequent core body temperature is less than or equal to 95 degrees Fahrenheit, a warning element.

20. The method of claim 13, further comprising:
receiving a signal indicating an environmental temperature;
determining whether the environmental temperature is equal to or greater than 150 degrees Fahrenheit; and
activating, responsive to determining the environmental temperature is equal to or greater than 150 degrees Fahrenheit, a warning element.
